(19) United States
(12) Patent Application Publication
(10) Pub. No.: US 2025/0140856 A1
(43) Pub. Date: May 1, 2025

(54) OXYGEN-BRIDGED GREEN ELECTRODES FROM PLANT-BASED BYPRODUCTS

(71) Applicant: Research Foundation of the City University of New York, New York, NY (US)

(72) Inventors: George John, Edison, NJ (US); Harrison Asare, West Haven, CT (US)

(21) Appl. No.: 18/927,291

(22) Filed: Oct. 25, 2024

Related U.S. Application Data
(60) Provisional application No. 63/592,950, filed on Oct. 25, 2023.

(51) Int. Cl. H01M 4/60; C07D 311/78; H01M 4/62

(52) U.S. Cl. CPC H01M 4/60; C07D 311/78; H01M 4/625

(57) ABSTRACT

An electrode material having a structure of Formula A

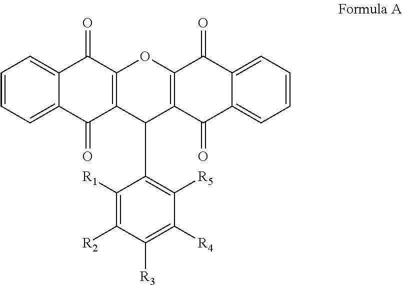

wherein R₁, R₂, R₃, R₄ and R₅ are H or Formula A-1

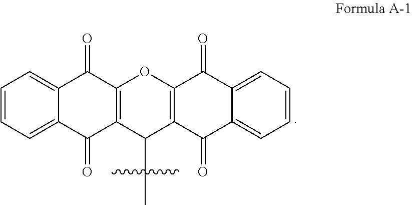 [SMILES: *C1C2=C(OC3=C1C(=O)c1ccccc1C3=O)C(=O)c1ccccc1C2=O]

### CROSS-REFERENCE TO RELATED APPLICATIONS

This application claims priority to, and is a non-provisional of, U.S. Patent Application 63/592,950 (filed Oct. 25, 2023), the entirety of which is incorporated herein by reference.

### STATEMENT OF FEDERALLY SPONSORED RESEARCH OR DEVELOPMENT

This invention was made with Government support under grant number 80NSSC19M0199 awarded by the National Aeronautics and Space Administration. The government has certain rights in the invention.

### BACKGROUND OF THE INVENTION

The subject matter disclosed herein relates to electrodes. Sustainable electrode materials are needed to serve the next generation of batteries to facilitate the shift towards clean energy storage and their usage. Organic materials are viable electrode alternatives to ensure the environment's well-being because the electrodes are low-cost, renewable, safe, and abundant. However, the organic materials undesirably dissolve in electrolyte leading to rapid capacity fading which causes low energy densities during cycling and impedes long cycling life of the battery. An improved electrode material is therefore desired.

The discussion above is merely provided for general background information and is not intended to be used as an aid in determining the scope of the claimed subject matter.

### SUMMARY

This disclosure provides an electrode material having a structure of Formula A.

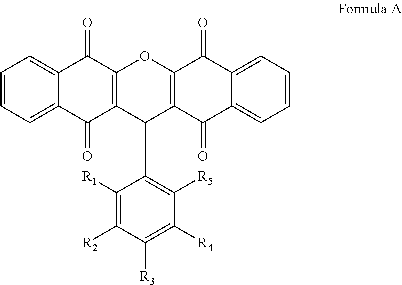

wherein R₁, R₂, R₃, R₄ and R₅ are each independently selected from the group consisting of H and Formula A-1.

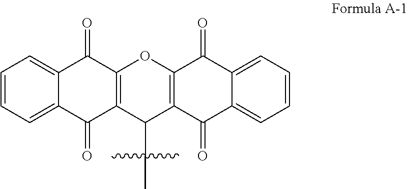 [SMILES: *C1C2=C(OC3=C1C(=O)c1ccccc1C3=O)C(=O)c1ccccc1C2=O]

An advantage that may be realized in the practice of some disclosed embodiments is that the electrode material is insoluble in liquid electrolytes at neural and basic pH.

In a first embodiment, a battery is provided. The battery comprising a first electrode comprising a compound of Formula A:

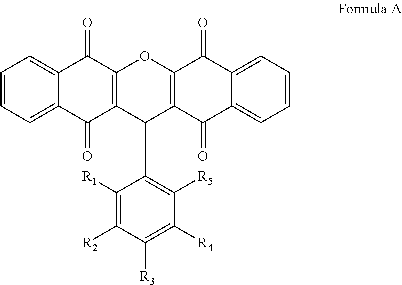

wherein R₁, R₂, R₃, R₄ and R₅ are each independently selected from the group consisting of H and Formula A-1

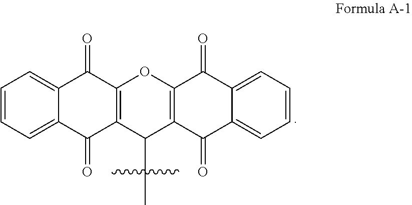 [SMILES: *C1C2=C(OC3=C1C(=O)c1ccccc1C3=O)C(=O)c1ccccc1C2=O]

In a second embodiment, a composition of matter is provided. The composition of matter consisting of a conductive material and a compound of Formula A:

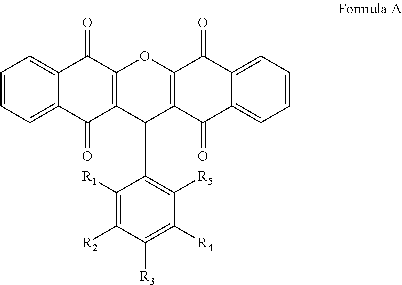

wherein R₁, R₂, R₃, R₄ and R₅ are each independently selected from the group consisting of H and Formula A-1

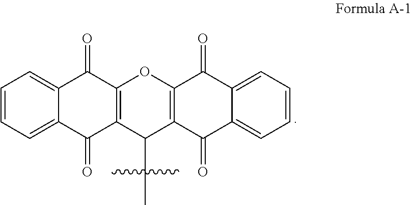 [SMILES: *C1C2=C(OC3=C1C(=O)c1ccccc1C3=O)C(=O)c1ccccc1C2=O]

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic depiction of a battery.

FIG. 2 illustrates the chemical structure of compound 200.

FIG. 3A is a graph depicting cyclic voltammetry (CV) curves of compound 200.

FIG. 3B is a graph resulting from a galvanostatic study.

FIG. 3C is a graph of specific capacity as a function of number of cycles.

FIG. 4 is an example of a synthetic scheme to produce compound 200.

### DETAILED DESCRIPTION OF THE INVENTION

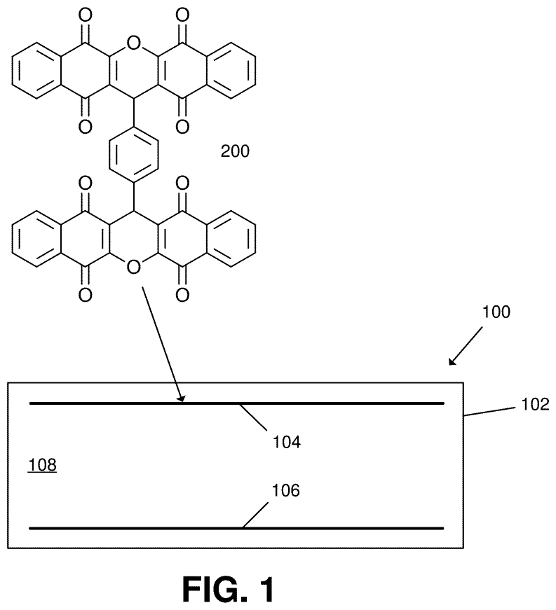

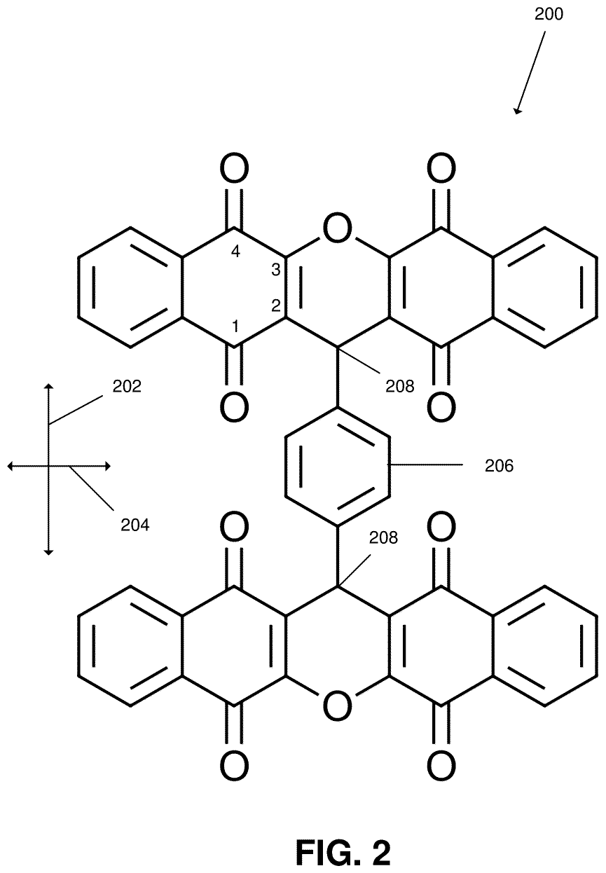

FIG. 1 schematically depicts a battery 100 that comprises an electrochemical cell 102, a first electrode 104, a second electrode 106 and an electrolyte 108. The first electrode 104 and the second electrode 106 form an anode-cathode pair. The first electrode 104 comprises compound 200, oxygen-bridged tetrakislawsone (OBTKL) (see FIG. 2).

Referring to FIG. 2, compound 200 is symmetric along a vertical 202 and a horizontal axis 204 around a para disubstituted benzene core 206. The benzene core 206 connects to tertiary carbons 208. The tertiary carbon 208 connects to two lawsone molecules at the two positions. Compounds 200 is yellowish-green in color, and demonstrates an innate insolubility in most solvents at neutral and basic pH. The insolubility makes compound 200 useful as an electrode material.

The first electrode 104 comprises compound 200 and a conductive material. Examples of conductive materials include conductive carbon. Examples of conductive carbon include C65 (e.g. sold by the MTI Corporation, SUPER P®, ketjen black, acetylene black (sold by the Denka Company Limited)). The compound 200 and the conductive material may be mixed to form a homogenous mixture and then formed into the first electrode 104 by a variety of conventional methods (e.g., dry pressing). The composition may be at least 50 wt %, at least 60 wt %, at least 70 wt %, at least 80 wt % or at least 90 wt % compound 200, with the balance being the conductive material. The shape of the first electrode 104 is not particularly limited and can be, for example, cylinders, discs (e.g., for use in coin cells), sheets, etc. The shape is a compressed solid. As used in this specification, a compressed solid is a solid that holds its shape under ambient conditions (22° C., 1 atm) when impacted by a force of 0.5N.

### Example

Electrode preparation was performed in an argon filled glovebox. An electrode was prepared by a dry pressing method. An electrode mixture was prepared by mixing compound 200 and a conductive carbon (C65 sold by the MTI Corporation) in a weight ratio of 60:40). The homogeneous mixture was pressed between two carbon papers. The resulting electrode was used to prepare coin cells in CR2032 format. Electrochemical analyses was performed in conventional coin cells, and the coin cells were prepared in an Ar filled glove box (O₂ less than 1 ppm, H₂O less than 0.1 ppm). Li foil (75 μm, Alfa Aesar) was used as anode and its surface was cleaned using razor blades, trilayer CELGARD® membrane was used as separator. Performance is reported in 1M lithium bis(trifluoromethanesulfonyl)imide (LiTFSI) and tetraethylene glycol dimethyl ether (tetraglyme) and 1M lithium hexafluorophosphate (LiPF6) in ethylene carbonate and dimethyl carbonate solvents. Galvanostatic tests were performed either on Arbin battery cycler or Landt instruments at appropriate current density. The specific capacity was calculated for electrodes based on the loading of compound 200 in the electrolyte. Cyclic voltametric studies were carried out using a biologic SP-300 potentiostat.

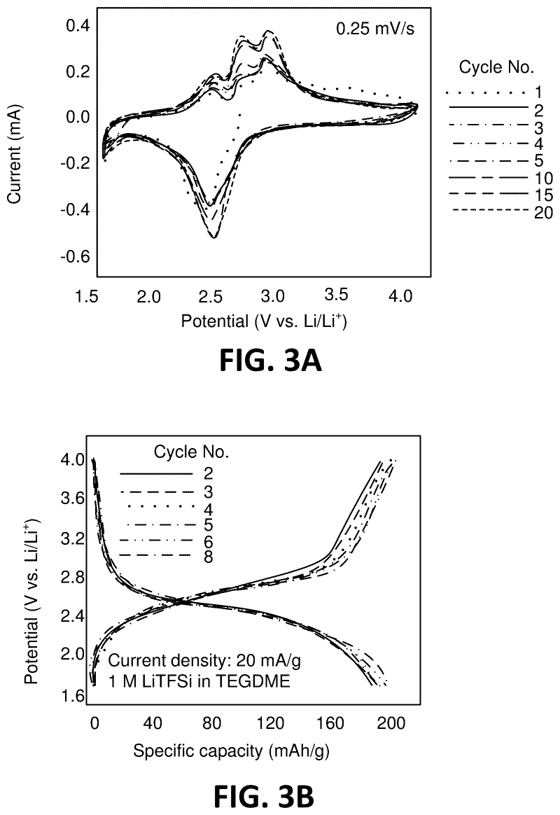

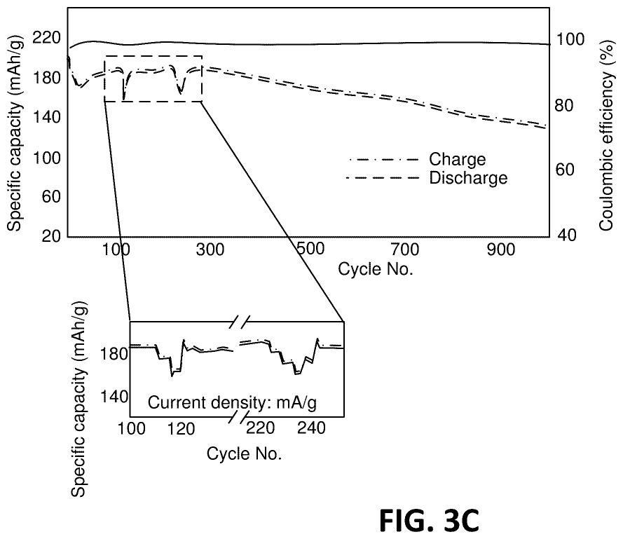

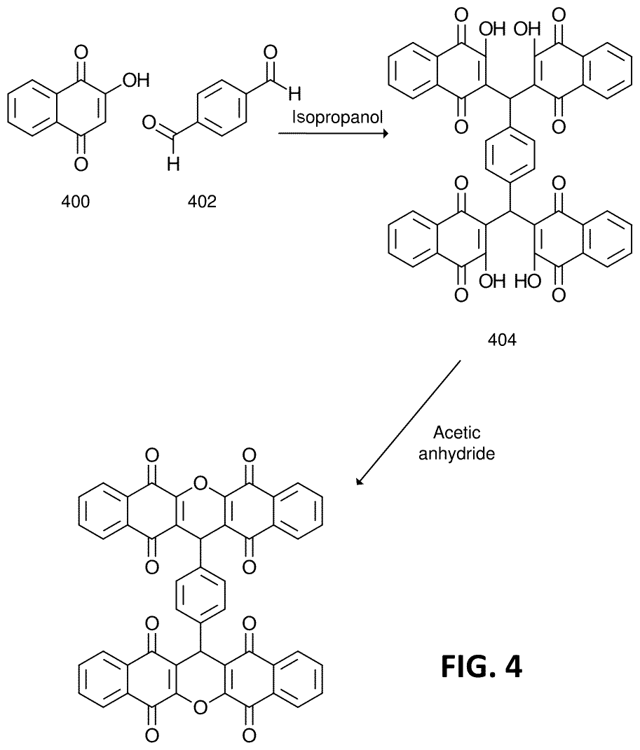

Referring to FIG. 3A, cyclic voltammetry (CV) curves of compound 200 reveal three prominent oxidation and two reduction peaks within the potential window of 1.6-4.1 V vs Li. The CV indicates the battery undergoes reversible oxidation and reduction electrochemical properties which shows the material is useful as a rechargeable battery.

Referring to FIG. 3B, galvanostatic studies confirm different redox reaction pathways as the voltage profiles of compound 200, in ether and carbonate electrolytes, show different line shapes and redox potentials.

Referring to FIG. 3C, in ether-based electrolyte, approximately 180 mAh per g of capacity was achieved before reaching 3 V.

Compound 200 in a Li-battery with the ether-based electrolyte demonstrates exceptional variable rate cycling capabilities and long-life stabilities. The Li-battery demonstrates excellent variable rate reversibility between 100 and 1000 mA per g and achieved the initial specific capacity at 100 mA per g (vide FIG. 3C insert). The Li-battery with compound 200 lasted over 1000 cycles while maintaining 70% of initial specific capacity, a significant improvement over conventional Li-organic batteries. Also, the Li-battery achieves a consistent 98% coulombic efficiency over 1000 cycle, implying charges stored are efficiently reproduced.

### Example of Synthesis of Compound 200

The synthesis uses a molecule that naturally occurs in the leaves of the henna plant called lawsone. Lawsone 400 (0.002 mol, 0.348 g, TCI America >99%) and terephthalaldehyde 402 (0.0005 mol, 0.067065 g, Acros >98%) were added to a round bottom flask followed with reagent-grade isopropanol (8 ml, VWR, >70%) and refluxed for 22 h. The reaction product 404 was generously washed with isopropanol during vacuum filtration and vacuum dried. Product 404 (1 g, 0.00126 mol) was refluxed in freshly purchased acetic anhydride (15 ml, Thermofisher Scientific, >99%) to form compound 200. The yellowish-greenish product was washed with acetone to remove excess acetic anhydride and vacuum-dried in an oven overnight.

In other embodiments, a different aldehyde can be used. Examples include isophthalaldehyde and other aldehydes like benzaldehyde.

The molecular structure of compound 200 was confirmed with multiple spectroscopic techniques including Fourier Transform Infrared (FTIR), solid-state Nuclear Magnetic Resonance (ssNMR) spectroscopy experiments like ¹³C cross polarization and ¹H single pulse acquisitions. Matrix assisted laser desorption/ionization experiments confirmed the expected molecular weight of 758 g per mol.

The insoluble nature of compound 200 is a beneficial trait that prevents dissolution in electrolyte, an improvement that ensure capacity retention in the battery. In one embodiment, the battery has a liquid electrolyte with a neutral or basic pH. In one embodiment, the electrolyte is an aqueous electrolyte with a neutral or basic pH (e.g. a pH equal to or greater than 7, 8, 9, 10, 11, 12, 13 or 14).

Further, compound 200 structurally is endowed with multiple carbonyl groups that are redox-active, owing to the reversible keto-enolate transformation during redox. Compound 200 undergoes n-type transformation by reducing from its pristine state to form an anion that coordinates to cations. Therefore, compound 200 as an electrode can be used metal-batteries and metal-ion batteries (e.g. Li, Na, Mg, Al, etc.). In addition to the carbonyl redox groups, the oxygen heteroatom contributes to the electrochemical properties of the battery by ensuring continuity of resonance, tuning the energy gaps of the frontier orbitals to improve electron transport, voltage tuning, and promote ion storage during charge compensation.

Referring again to Formula A, R₁, R₂, R₃, R₄ and R₅ are each independently selected from the group consisting of H and Formula A-1. In compound 200, R₁, R₂, R₄ and R₅ are hydrogens and R₃ is Formula A-1. In other embodiments, R₁, R₂, R₃, R₄ and R₅ include four hydrogens and one Formula A-1. In another embodiment, R₁, R₂, R₃, R₄ and R₅ are all hydrogen.

Additionally, Lawsone 400 has demonstrated antifungal capabilities, therefore compound 200 is believed to have similar pharmaceutical capabilities.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A battery comprising a first electrode comprising a compound of Formula A: 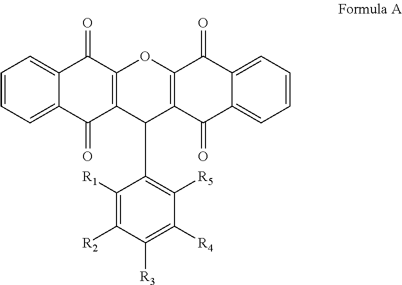
wherein R₁, R₂, R₃, R₄ and R₅ are each independently selected from the group consisting of H and Formula A-1

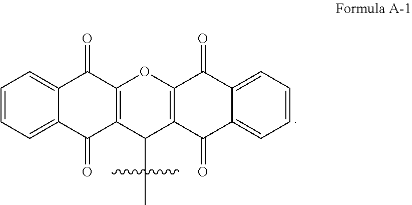 [SMILES: *C1C2=C(OC3=C1C(=O)c1ccccc1C3=O)C(=O)c1ccccc1C2=O]

2. The battery as recited in claim 1, the first electrode further comprising a conductive material.

3. The battery as recited in claim 2, wherein the conductive material is conductive carbon.

4. The battery as recited in claim 2, wherein the first electrode consists of the compound and the conductive material.

5. The battery as recited in claim 2, wherein the first electrode is a cathode and the battery further comprises a second electrode that is an anode.

6. The battery as recited in claim 5, wherein the second electrode is a lithium electrode, a sodium electrode, a magnesium electrode or an aluminum electrode.

7. The battery as recited in claim 5, wherein the second electrode is a lithium electrode.

8. The battery as recited in claim 5, wherein the battery further comprises an aqueous electrolyte with a pH greater than or equal to 7.

9. The battery as recited in claim 5, wherein the battery further comprises an aqueous electrolyte with a pH greater than or equal to 8.

10. The battery as recited in claim 5, wherein the battery further comprises an aqueous electrolyte with a pH greater than or equal to 9.

11. The battery as recited in claim 5, wherein the battery further comprises an aqueous electrolyte with a pH greater than or equal to 10.

12. The battery as recited in claim 5, wherein the battery further comprises an aqueous electrolyte with a pH greater than or equal to 11.

13. The battery as recited in claim 5, wherein the battery further comprises an aqueous electrolyte with a pH greater than or equal to 12.

14. The battery as recited in claim 2, wherein R₁, R₂, R₃, R₄ and R₅ include four hydrogens and one Formula A-1.

15. The battery as recited in claim 2, wherein R₁, R₂, R₄ and R₅ are hydrogens and R₃ is Formula A-1.

16. The battery as recited in claim 2, wherein R₁, R₂, R₃, R₄ and R₅ are hydrogens.

17. A composition of matter consisting of a conductive material and a compound of Formula A: 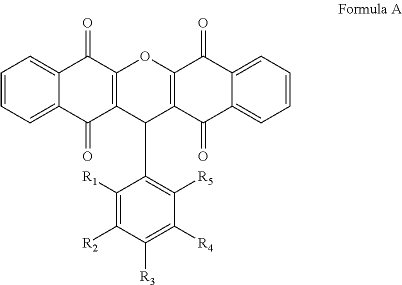
wherein R₁, R₂, R₃, R₄ and R₅ are each independently selected from the group consisting of H and Formula A-1

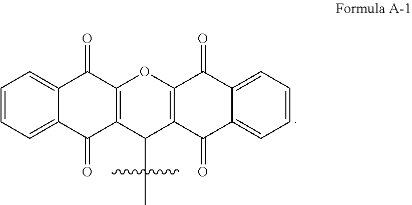 [SMILES: *C1C2=C(OC3=C1C(=O)c1ccccc1C3=O)C(=O)c1ccccc1C2=O]

18. The composition of matter as recited in claim 17, wherein the conductive material is conductive carbon.

19. The composition of matter as recited in claim 18, wherein the composition of matter is a compressed solid.

20. The composition of matter as recited in claim 19, wherein R₁, R₂, R₄ and R₅ are hydrogens and R₃ is Formula A-1.
